(19) United States
(12) Patent Application Publication
(10) Pub. No.: US 2026/0191903 A1
(43) Pub. Date: Jul. 9, 2026

(54) FREEZE-DRIED PLASMA (FDP) FOR WELL-BALANCED COAGULATION AND FIBRINOLYSIS, METHOD OF PREPARATION AND USE THEREOF

(71) Applicant: ARMY MEDICAL UNIVERSITY, PEOPLE'S LIBERATION ARMY, PRC, Chongqing (CN)

(72) Inventors: Zhaowen ZONG, Chongqing (CN); Wenqiong DU, Chongqing (CN); Chenglin DAI, Chongqing (CN); Lin CHEN, Chongqing (CN); Can CHEN, Chongqing (CN); Yijun JIA, Chongqing (CN); Renqing JIANG, Chongqing (CN); Xin ZHONG, Chongqing (CN); Haoyang YANG, Chongqing (CN); Song LIU, Chongqing (CN)

(73) Assignee: ARMY MEDICAL UNIVERSITY, PEOPLE'S LIBERATION ARMY, PRC, Chongqing (CN)

(21) Appl. No.: 19/058,508

(22) Filed: Feb. 20, 2025

(30) Foreign Application Priority Data
Jan. 8, 2025 (CN) 202510031432.7

(51) Int. Cl. A61K 35/16; A61K 38/36; A61K 38/48; A61K 38/57

(52) U.S. Cl. CPC A61K 35/16; A61K 38/36; A61K 38/363; A61K 38/4833; A61K 38/484; A61K 38/57; C12Y 304/21005; C12Y 304/21007

(57) ABSTRACT

A freeze-dried plasma (FDP) for well-balanced coagulation and fibrinolysis, a method of preparation and use thereof are provided, relating to the technical field of hemostatic preparations. The FDP for well-balanced coagulation and fibrinolysis is compounded with appropriate concentrations of coagulation factors, anticoagulant factors, fibrinolytic factors, fibrinolysis inhibiting factors, and von Willebrand factor according to different changes in a coagulation-fibrinolysis system after different types of tissue and organ injuries. The FDP for well-balanced coagulation and fibrinolysis is used for hemostatic resuscitation of general severe trauma, severe hepatic trauma, severe craniocerebral injury, and severe trauma with seawater immersion and other types of injuries, exhibiting a desirable hemostatic resuscitation effect. It does not cause thrombosis in uninjured parts, and may reduce the amplitude of lactate increase after the trauma.

### CROSS REFERENCE TO RELATED APPLICATION

This application claims foreign priority benefits under 35 U.S.C. § 119(a)-(d) to Chinese Patent Application No. 202510031432.7 filed on Jan. 8, 2025, which is hereby incorporated herein by reference in its entirety.

### TECHNICAL FIELD

A freeze-dried plasma (FDP) with well-balanced coagulation and fibrinolysis, a method of preparation, and use thereof are provided, relating to the technical field of hemostatic preparations.

### BACKGROUND

Freeze-dried plasma (FDP) is widely used in pre-hospital and wartime treatment in many countries, showing a desirable resuscitation effect. However, currently used FDP products exhibit a significant decrease in multiple coagulation factors during the freeze-drying process, which limits their efficacy for pre-hospital hemostatic resuscitation. During the freeze-drying and storage, some coagulation factors in FDP lose their activity, resulting in a significant decrease in von Willebrand factor (vWF) and coagulation factors II, V, VII, VIII, and X. When patients with severe trauma develop traumatic coagulopathy, the reduction in their coagulation factors and other factors significantly exceeds that in a level of related factors in the lost blood. The changes in their coagulation-fibrinolysis system vary according to the different types of tissue and organ injuries. These factors contribute to the inability of conventional FDP to achieve the hemostatic resuscitation goals and effects required for patients with severe trauma.

Current studies have shown that FDP exhibits better resuscitation effects in trauma patients. A meta-analysis by Feuerstein et al. [Feuerstein S J, et al. Freeze-dried plasma in major hemorrhage: a systematic review. Vox Sang. 2020; 115(4): 263-74.] has shown that there is no significant difference in mortality 30 d after injury between FDP and fresh frozen plasma (FFP). In 2022, a meta-analysis by Mok et al. [Mok G, et al. Freeze-dried plasma for major trauma-Systematic review and meta-analysis. J Trauma Acute Care Surg. 2021; 90(3): 589-602.] showed that there was no significant difference in mortality between trauma patients receiving FDP and those receiving FFP. Animal experimental results have indicated that there is no difference in coagulation factor levels and anti-inflammatory properties between FDP and FFP. However, the clinical observation of the above literature primarily focused on mortality, without assessing indicators such as coagulation function, lactate, and organ function in trauma patients, and was limited to comparing the differences between FDP and FFP.

In 2024, Sheffield conducted a meta-analysis on the effectiveness of FDP in trauma patients, including three high-level randomized controlled studies. A clinical study, abbreviated as RePHILL, included 501 trauma patients in the UK, evaluating mortality and lactate clearance rate, with the control group receiving resuscitated with normal saline. The results showed no statistical difference in the mortality and lactate clearance rate between the two groups. Another study, named as PREHO-PLYO, included 150 patients randomly assigned to the hospital to receive up to 4 units of FDP or up to 1,000 mL of normal saline. The results showed no significant difference in the international normalized ratio (INR) between the two groups. The third study evaluated the feasibility of transfusing FDP and red blood cells in the prehospital setting of the Australian College of Aeronautical Medicine using a randomized controlled design, and found that the FDP group may have a lower mortality at 24 h post-admission and at discharge [Sheffield W P, et al. Prehospital Freeze-Dried Plasma in Trauma: A Critical Review. Transfus Med Rev. 2024; 38(1): 150807.].

In summary, although there is no significant difference in mortality between FDP and FFP, FDP does not appear to improve the coagulation state and shock correction compared with normal saline in injured patients. More importantly, the above studies did not stratify trauma patients according to injury severity. The coagulation-anticoagulation-fibrinolysis-antifibrinolysis system disorder in severely traumatized patients is more serious and complicated than that in mildly traumatized patients, and the traditional FDP resuscitation may not achieve the goal of hemostatic resuscitation strategy.

### SUMMARY

In order to solve the above problems, the present disclosure provides an FDP for well-balanced coagulation and fibrinolysis, a method of preparation and use thereof. The FDP for well-balanced coagulation and fibrinolysis is used for hemostatic resuscitation of general severe trauma, liver-dominant severe trauma, severe craniocerebral injury, and severe trauma combined with seawater immersion and other types of injuries, and has a desirable hemostatic resuscitation effect.

To achieve the above objective, the present disclosure provides the following technical solutions:

The present disclosure provides an FDP for well-balanced coagulation and fibrinolysis, including the following components in parts by mass: an FDP, 32 parts to 600 parts of coagulation factor II, 33.5 parts to 1,200 parts of fibrinogen, 0.7 parts to 16 parts of coagulation factor V, 0.9 parts to 16 parts of coagulation factor X, 3.1 parts to 50 parts of antithrombin III, 0 parts to 24 parts of plasminogen, plasminogen activation inhibitor-1, and 0.112 parts to 2.1 parts of von Willebrand factor; where the ratio of a mass of coagulation factor II to an enzyme activity of the plasminogen activation inhibitor-1 is 32-600 mg: 104-4000 AU; and assuming the FDP prepared from 200 mL of a human plasma or 20 mL of a rabbit plasma is recorded as 1 unit, the FDP and the coagulation factor II are at a ratio of 1 unit: (32-600) mg.

In some embodiments, the FDP for well-balanced coagulation and fibrinolysis is one or more selected from the group consisting of independently packaged:
preparation 1, including the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; where the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2, including the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; where the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3, including plasminogen activation inhibitor-1, and the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; where the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4, including the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; where the ratio of a mass of coagulation factor II to an enzyme activity of the plasminogen activation inhibitor-1 is 300 mg: 4000 AU; and the FDP and the coagulation factor II are at a ratio of 1 unit: 300 mg;
where the FDP in the preparation 1 to the preparation 4 each is human FDP;
preparation 5, including the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; where the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6, including the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; where the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7, including the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; where the ratio of a mass of the coagulation factor II to an enzyme activity of the plasminogen activation inhibitor-1 is 32 mg: 416 AU; and the FDP and the coagulation factor II are at a ratio of 1 unit: 32 mg; and
preparation 8 including the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; where the ratio of a mass of coagulation factor II to an enzyme activity of the plasminogen activation inhibitor-1 is 32 mg: 416 AU; and the FDP and the coagulation factor II are at a ratio of 1 unit: 32 mg;
where the FDP in the preparation 5 to the preparation 8 each is rabbit FDP.

In some embodiments, a raw material of the FDP in the preparation 1 is selected from the group consisting of fresh human plasma and human platelet-rich plasma (PRP); and a raw material of the FDP in the preparation 5 is selected from the group consisting of fresh rabbit plasma and rabbit PRP.

In some embodiments, a raw material of the FDP in preparation 2 to preparation 4 is fresh human plasma; and a raw material of the FDP in the preparation 6 to the preparation 8 is fresh rabbit plasma.

The present disclosure further provides a method for preparing the FDP for well-balanced coagulation and fibrinolysis, including: mixing the components in the parts by mass to obtain the FDP for well-balanced coagulation and fibrinolysis.

In some embodiments, a preparation process of the FDP in the FDP for well-balanced coagulation and fibrinolysis includes the following steps:
mixing a plasma, ascorbic acid, and glycine to obtain a primary plasma;
subjecting the primary plasma to pre-freezing at −40° C. for 2 h, vacuumizing to not greater than 30 Pa, and then freeze-drying at −40° C. for 10 h; and
subjecting a product obtained by the freeze-drying to vacuum-drying at −35° C. for 6 h such that a moisture content of the product is not greater than 3% to obtain the FDP.

In some embodiments, the primary plasma includes the ascorbic acid at a concentration of 2 mmol/L to 4 mmol/L and the glycine at a concentration of 60 mmol/L.

The present disclosure further provides use of the FDP for well-balanced coagulation and fibrinolysis or an FDP for well-balanced coagulation and fibrinolysis prepared by the preparation method in preparation of a hemostatic product.

In some embodiments, the hemostatic product is a hemostatic resuscitation product.

In some embodiments, the hemostatic resuscitation product is used for one or more selected from the group consisting of general severe trauma, liver-dominant severe trauma, severe craniocerebral injury, and severe trauma combined with seawater immersion.

### Beneficial Effects

The FDP for well-balanced coagulation and fibrinolysis is compounded with appropriate concentrations of coagulation factors, anticoagulant factors, fibrinolytic factors, fibrinolysis inhibiting factors, and von Willebrand factor according to different changes in a coagulation-fibrinolysis system after different types of tissue and organ injuries. The FDP for well-balanced coagulation and fibrinolysis may be used for hemostatic resuscitation of general severe trauma, severe hepatic trauma, severe craniocerebral trauma, severe trauma with seawater immersion and other types of injuries, with desirable hemostatic resuscitation effect. It does not cause thrombosis in uninjured parts, and may reduce the amplitude of lactate increase after the trauma.

### BRIEF DESCRIPTION OF THE DRAWINGS

To illustrate the embodiments of the present disclosure or the technical solutions in the prior art more clearly, the accompanying drawings required in the examples will be briefly introduced below:

FIG. 1 shows the R values of each group in an in vitro experiment of Test Example 1;

FIG. 2 shows the α angle values of each group in an in vitro experiment of Test Example 1;

FIG. 3 shows the maximum amplitude (MA) values of each group in an in vitro experiment of Test Example 1;

FIG. 4 shows the R values of each group in an in vivo experiment of Test Example 1;

FIG. 5 shows the α angle values of each group in an in vivo experiment of Test Example 1;

FIG. 6 shows the MA values of each group in an in vivo experiment of Test Example 1;

FIG. 7 shows the blood loss of each group in an in vivo experiment in Test Example 1;

FIG. 8 shows the changes in lactate concentrations of each group in an in vivo experiment in Test Example 1;

FIG. 9 shows the animal lung tissue sections (×200) of the injury severity score (ISS) 18 group+2.0-fold FDP for well-balanced coagulation and fibrinolysis in Test Example 1;

FIG. 10 shows the animal liver tissue sections (×200) of the ISS 18 group+2.0-fold FDP for well-balanced coagulation and fibrinolysis in Test Example 1;

FIG. 11 shows the animal kidney tissue sections (×200) of the ISS 18 group+2.0-fold FDP for well-balanced coagulation and fibrinolysis in Test Example 1;

FIG. 12 shows the R values of each group in an in vitro experiment of Test Example 2;

FIG. 13 shows the α angle values of each group in an in vitro experiment of Test Example 2;

FIG. 14 shows the MA values of each group in an in vitro experiment of Test Example 2;

FIG. 15 shows the R values of each group in an in vivo experiment of Test Example 2;

FIG. 16 shows the α angle values of each group in an in vivo experiment of Test Example 2;

FIG. 17 shows the MA values of each group in an in vivo experiment of Test Example 2;

FIG. 18 shows the blood loss of each group in an in vivo experiment in Test Example 2;

FIG. 19 shows the changes in lactate concentrations of each group in an in vivo experiment in Test Example 2;

FIG. 20 shows the animal lung tissue sections (×200) of the fibrinogen-rich FDP for well-balanced coagulation and fibrinolysis in Test Example 2;

FIG. 21 shows the animal liver tissue sections (×200) of the fibrinogen-rich FDP for well-balanced coagulation and fibrinolysis in Test Example 2;

FIG. 22 shows the animal kidney tissue sections (×200) of the fibrinogen-rich FDP for well-balanced coagulation and fibrinolysis in Test Example 2;

FIG. 23 shows the R values of each group in an in vitro experiment of Test Example 3;

FIG. 24 shows the α angle values of each group in an in vitro experiment of Test Example 3;

FIG. 25 shows the MA values of each group in an in vitro experiment of Test Example 3;

FIG. 26 shows the R values of each group in an in vivo experiment of Test Example 3;

FIG. 27 shows the α angle values of each group in an in vivo experiment of Test Example 3;

FIG. 28 shows the MA values of each group in an in vivo experiment of Test Example 3;

FIG. 29 shows the blood loss of each group in an in vivo experiment in Test Example 3;

FIG. 30 shows the changes in lactate concentrations of each group in an in vivo experiment in Test Example 3;

FIG. 31 shows the animal lung tissue sections (×200) of the fibrinolysis inhibiting factor-rich FDP for well-balanced coagulation and fibrinolysis in Test Example 3;

FIG. 32 shows the animal liver tissue sections (×200) of the fibrinolysis inhibiting factor-rich FDP for well-balanced coagulation and fibrinolysis in Test Example 3;

FIG. 33 shows the animal kidney tissue sections (×200) of the fibrinolysis inhibiting factor-rich FDP for well-balanced coagulation and fibrinolysis in Test Example 3;

FIG. 34 shows the R values of each group in an in vitro experiment of Test Example 4;

FIG. 35 shows the α angle values of each group in an in vitro experiment of Test Example 4;

FIG. 36 shows the MA values of each group in an in vitro experiment of Test Example 4;

FIG. 37 shows the R values of each group in an in vivo experiment of Test Example 4;

FIG. 38 shows the α angle values of each group in an in vivo experiment of Test Example 4;

FIG. 39 shows the MA values of each group in an in vivo experiment of Test Example 4;

FIG. 40 shows the blood loss of each group in an in vivo experiment in Test Example 4;

FIG. 41 shows the changes in lactate concentrations of each group in an in vivo experiment in Test Example 4;

FIG. 42 shows the animal lung tissue sections (×200) of the platelet-rich FDP for well-balanced coagulation and fibrinolysis in Test Example 4;

FIG. 43 shows the animal liver tissue sections (×200) of the platelet-rich FDP for well-balanced coagulation and fibrinolysis in Test Example 4; and

FIG. 44 shows the animal kidney tissue sections (×200) of the platelet-rich FDP for well-balanced coagulation and fibrinolysis in Test Example 4; where

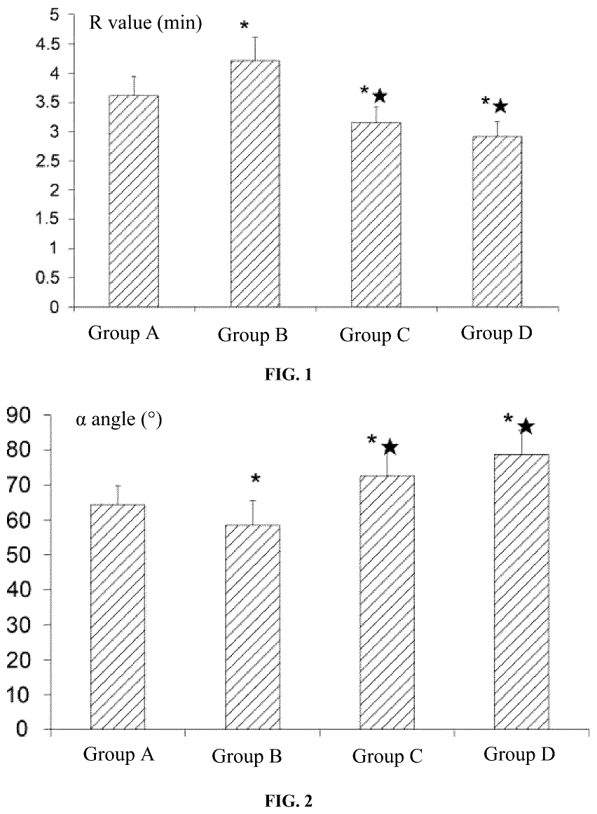

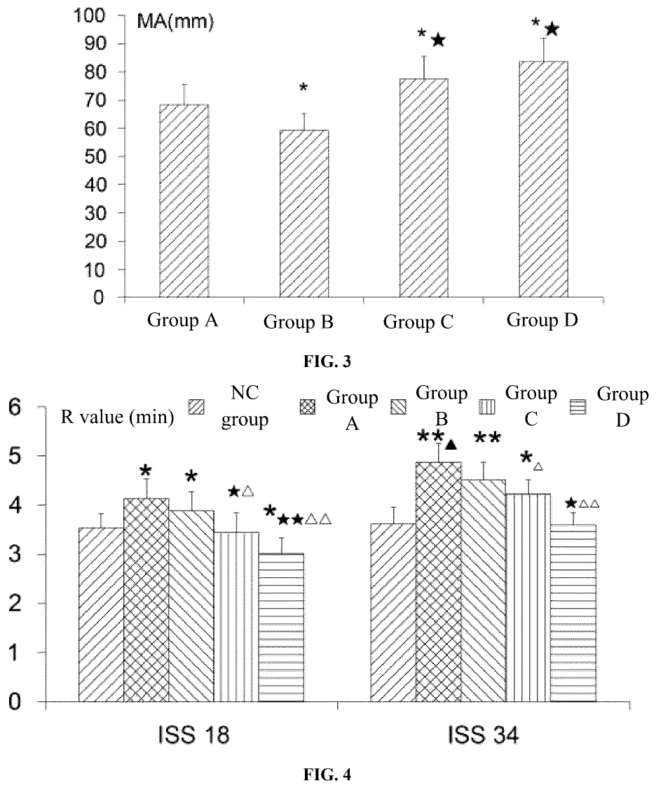

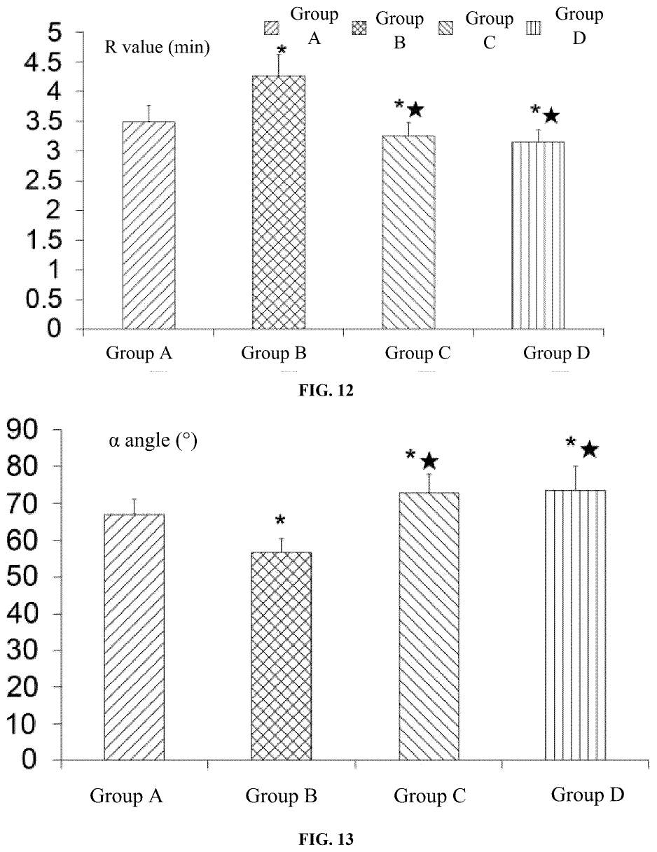

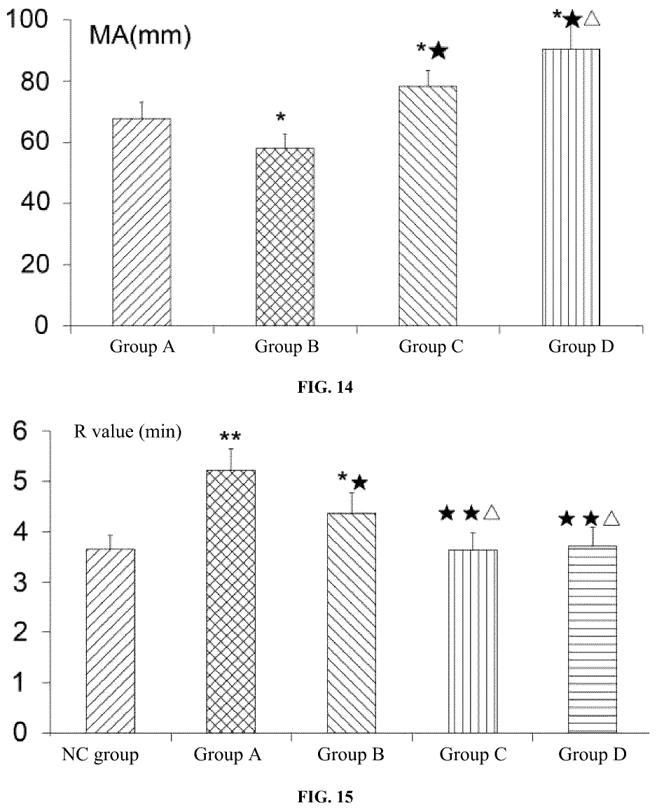

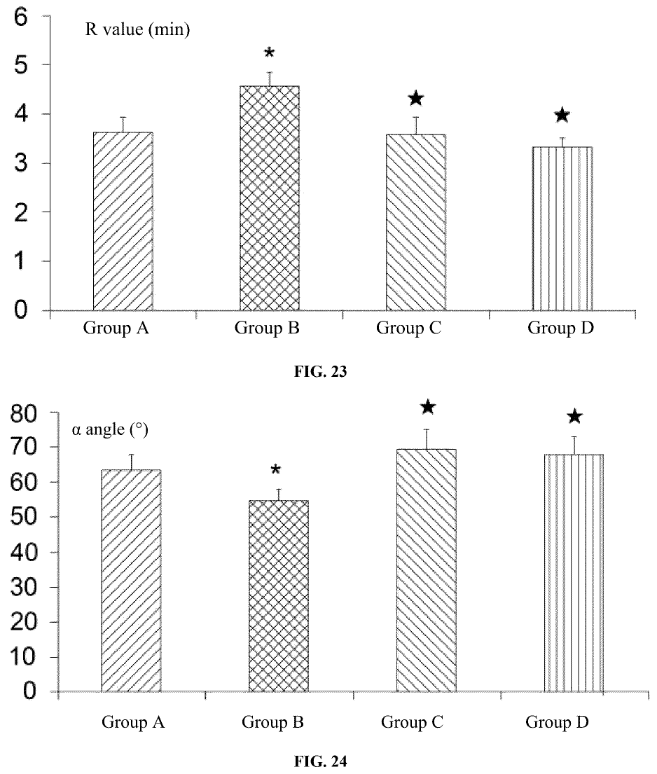

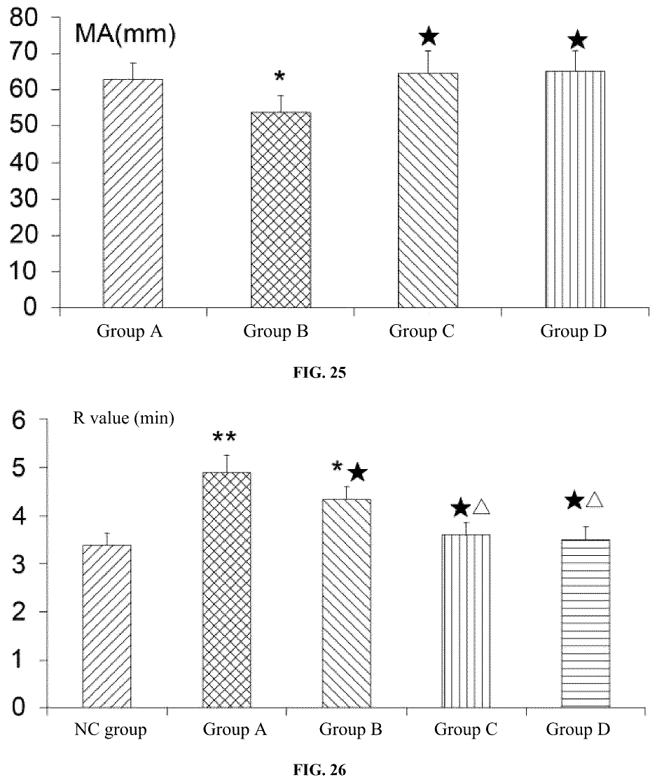

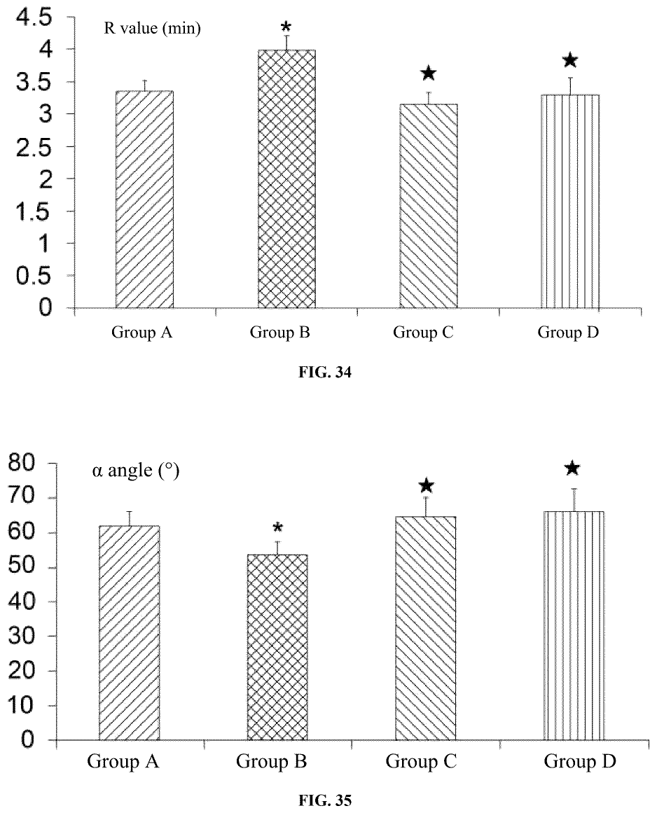

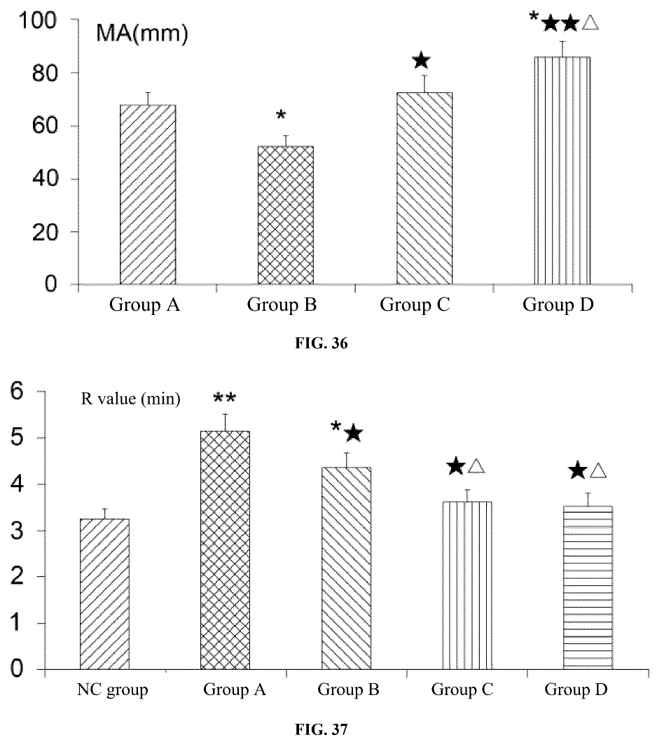

in FIG. 1 to FIG. 3, FIG. 12 to FIG. 14, FIG. 23 to FIG. 25, and FIG. 34 to FIG. 36, * indicates P<0.05 compared with Group A; ★ indicates P<0.05 compared with Group B; Δ indicates P<0.05 compared with Group C;

in FIG. 4 to FIG. 6 and FIG. 8, * indicates P<0.05 compared with Group NC having the same injury severity score (ISS), ** indicates P<0.01 compared with Group NC having the same ISS; Δ indicates P<0.05 compared with Group A having the same ISS, ΔΔ indicates P<0.01 compared with Group A having the same ISS; ★ indicates P<0.05 compared with Group B having the same ISS, ★★ indicates P<0.01 compared with Group B having the same ISS; ▴ indicates P<0.05 compared with the same treatment measure in the same injury model;

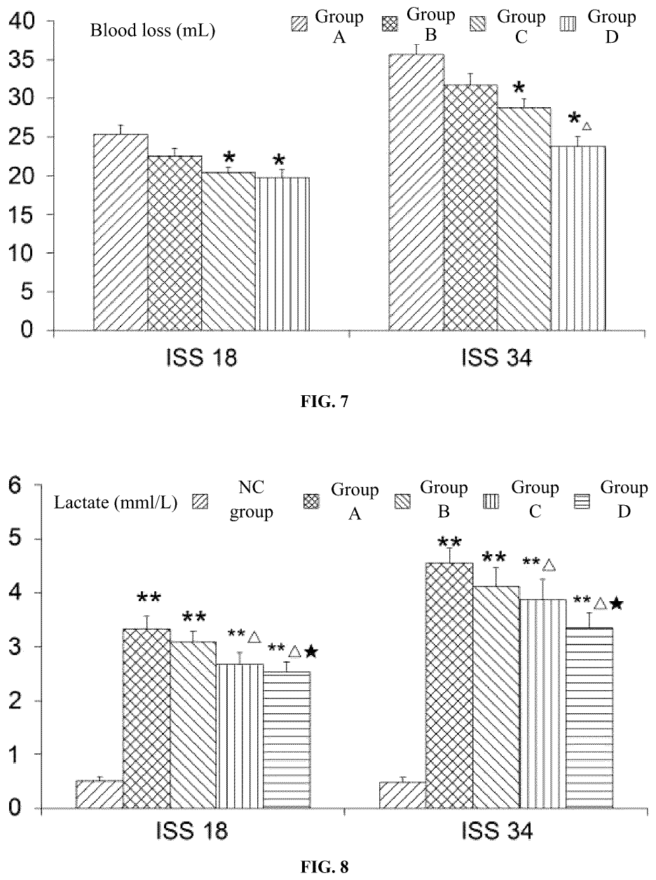

in FIG. 7, * indicates P<0.05 compared with Group A having the same ISS; Δ indicates P<0.05 compared with Group B having the same ISS;

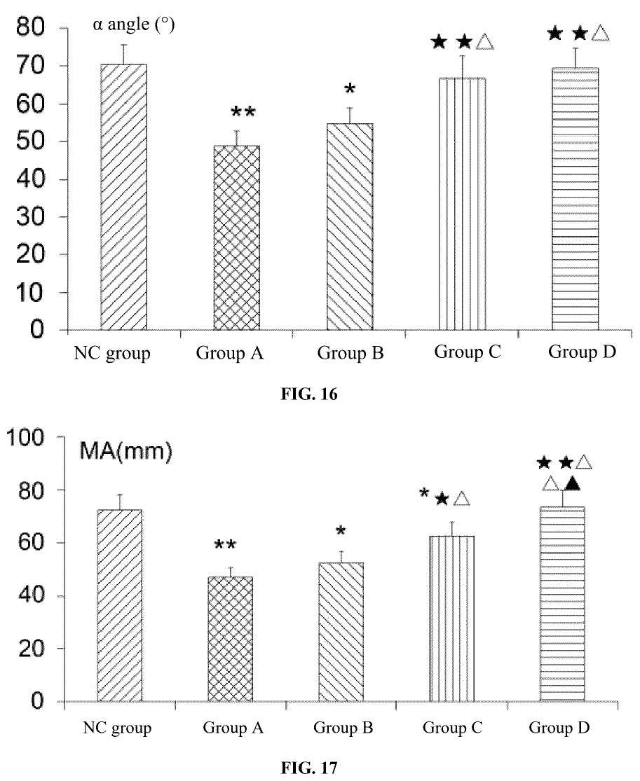

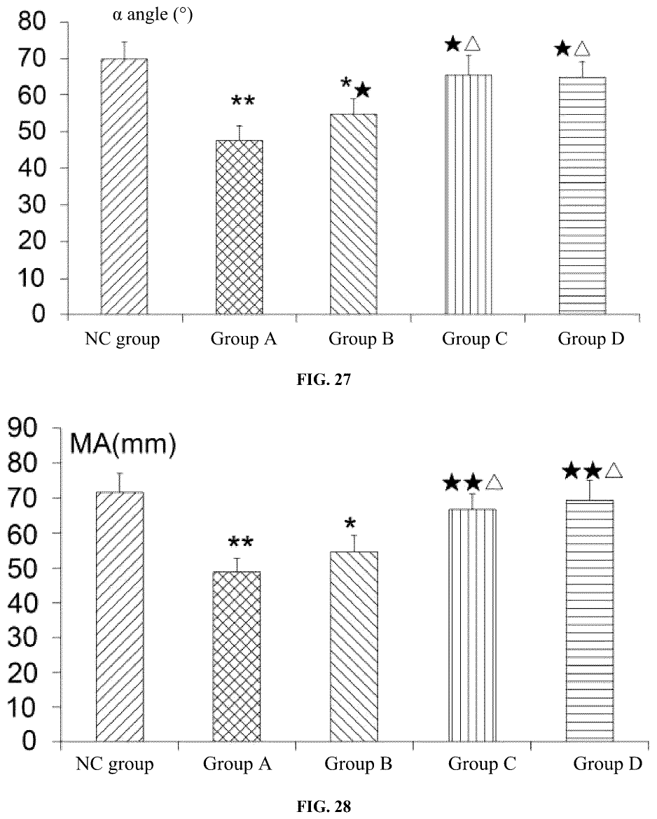

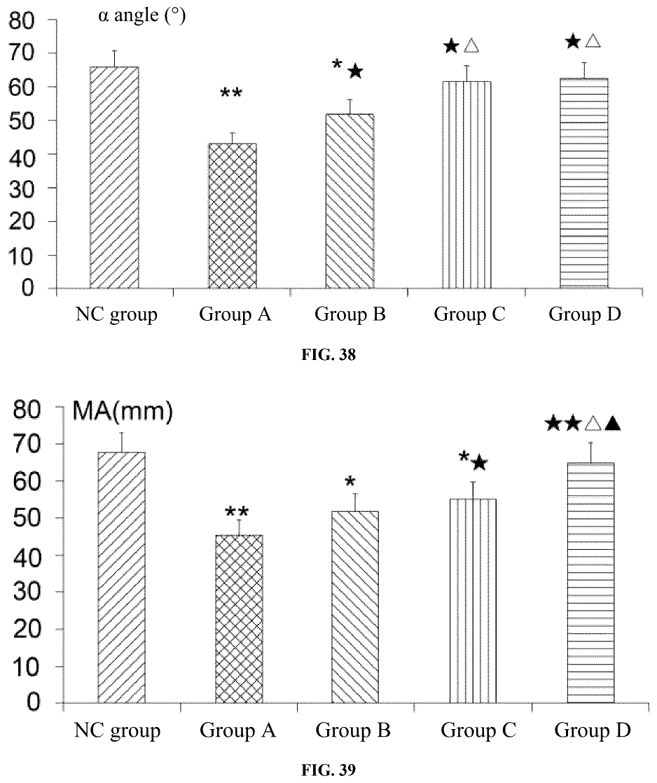

in FIG. 15 to FIG. 17, FIG. 19, FIG. 26 to FIG. 28, and FIG. 37 to FIG. 39, * indicates P<0.05 compared with Group NC, ** indicates P<0.01 compared with Group NC; ★ indicates P<0.05 compared with Group A, ★★ indicates P<0.01 compared with Group A; A indicates P<0.05 compared with Group B; ▴ indicates P<0.05 compared with Group C;

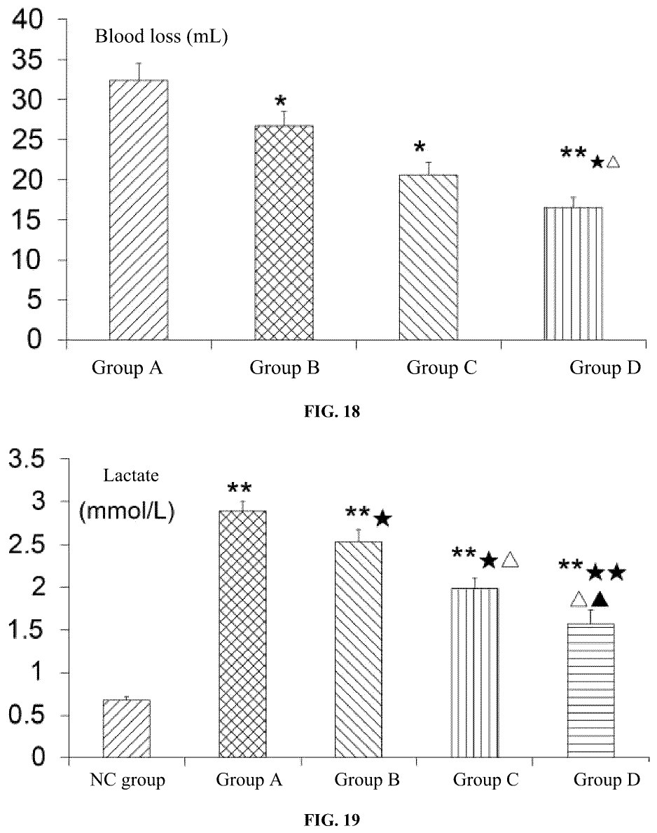

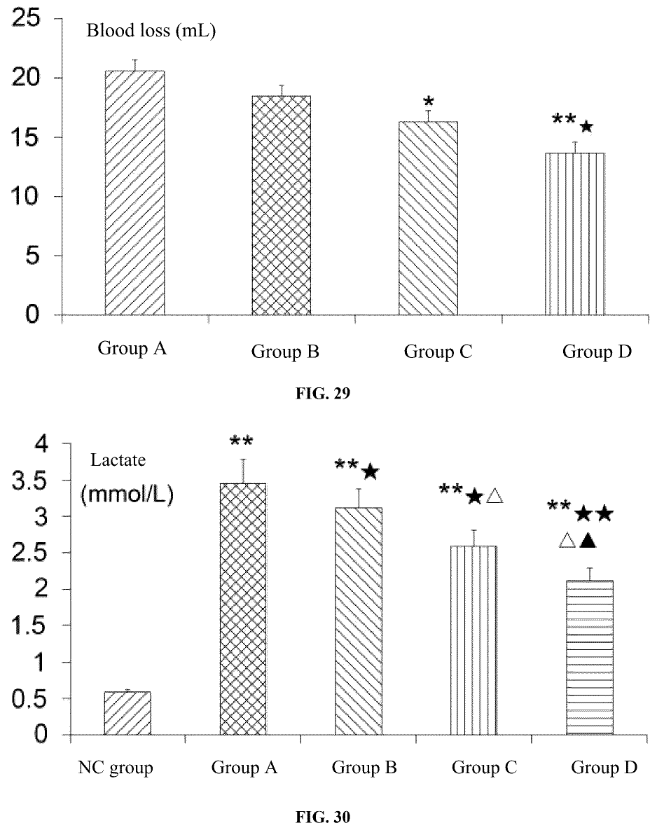

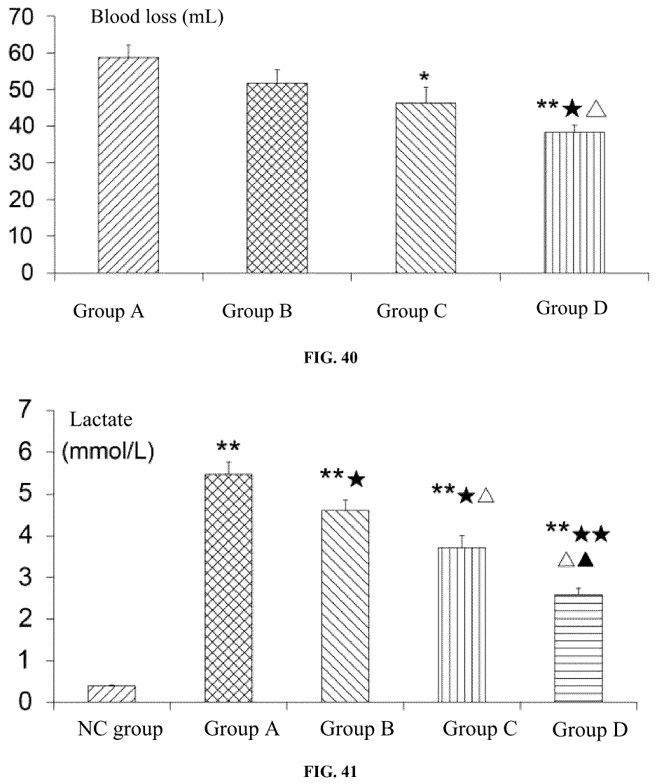

in FIG. 18, FIG. 29, and FIG. 40, * indicates P<0.05 compared with Group A, ** indicates P<0.01 compared with Group A; ★ indicates P<0.05 compared with Group B; Δ indicates P<0.05 compared with Group C; and

in FIG. 19, FIG. 30, and FIG. 41, ** indicates P<0.01 compared with Group NC; ★ indicates P<0.05 compared with Group A, ★★ indicates P<0.01 compared with Group A; A indicates P<0.05 compared with Group B; ▴ indicates P<0.05 compared with Group C.

### DETAILED DESCRIPTION

The present disclosure provides an FDP for well-balanced coagulation and fibrinolysis, including the following components in parts by mass: an FDP, 32 parts to 600 parts of coagulation factor II, 33.5 parts to 1,200 parts of fibrinogen, 0.7 parts to 16 parts of coagulation factor V, 0.9 parts to 16 parts of coagulation factor X, 3.1 parts to 50 parts of antithrombin III, 0 parts to 24 parts of plasminogen, plasminogen activation inhibitor-1, and 0.112 parts to 2.1 parts of von Willebrand factor; where the ratio of a mass of coagulation factor II to an enzyme activity of the plasminogen activation inhibitor-1 is (32-600) mg: (104-4000) AU; and assuming the FDP prepared from 200 mL of a human plasma or 20 mL of a rabbit plasma is recorded as 1 unit, the FDP and the coagulation factor II are at a ratio of 1 unit: (32-600) mg.

As an embodiment, the FDP for well-balanced coagulation and fibrinolysis is one or more selected from the group consisting of independently packaged
preparation 1 including the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; where the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2 including the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; where the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3 including the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; where the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4 including the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; where the ratio of a mass of coagulation factor II to an enzyme activity of the plasminogen activation inhibitor-1 is 300 mg: 4000 AU; and the FDP and the coagulation factor II are at a ratio of 1 unit: 300 mg;
where the FDP in preparation 1 to preparation 4 each is human FDP;
preparation 5 including the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; where the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6 including the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; where the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7 including the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; where the ratio of a mass of coagulation factor II to an enzyme activity of the plasminogen activation inhibitor-1 is 32 mg: 416 AU; and the FDP and the coagulation factor II are at a ratio of 1 unit: 32 mg; and
preparation 8 including the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; where the ratio of a mass of coagulation factor II to an enzyme activity of the plasminogen activation inhibitor-1 is 32 mg: 416 AU; and the FDP and the coagulation factor II are at a ratio of 1 unit: 32 mg;
where the FDP in preparation 5 to preparation 8 each is rabbit FDP.

As an embodiment, a raw material of the FDP in preparation 1 is selected from the group consisting of fresh human plasma and human PRP; and a raw material of the FDP in preparation 5 is selected from the group consisting of fresh rabbit plasma and rabbit PRP. As another embodiment, when the raw material of the FDP in preparation 1 is fresh human plasma, preparation 1 is recorded as preparation 1-1; when the raw material of the FDP in preparation 1 is human PRP, preparation 1 is recorded as preparation 1-2; when the raw material of the FDP in preparation 5 is fresh rabbit plasma, preparation 5 is recorded as preparation 5-1; when the raw material of the FDP in preparation 5 is rabbit PRP, preparation 5 is recorded as preparation 5-2.

As another embodiment, a raw material of the FDP in preparation 2 to preparation 4 is fresh human plasma; and a raw material of the FDP in preparation 6 to preparation 8 is fresh rabbit plasma.

During the resuscitation, increasing the content of coagulation factors in FDP helps to achieve the goal of hemostatic resuscitation. However, coagulation-anticoagulation-fibrinolysis-antifibrinolysis is in a dynamic equilibrium, and increased coagulation may increase the possibility of ectopic thrombus formation. The sterilization, lyophilization, and storage of FDP can affect the activity of coagulation-related factors. Compared with normal plasma, vWF and coagulation factors II, V, VII, and VIII have decreased most significantly in FDP. In the present disclosure, coagulation factors, anticoagulant factors, fibrinolytic factors, fibrinolysis inhibiting factors, and von Willebrand factor at appropriate concentrations are compounded in FDP. The FDP for well-balanced coagulation and fibrinolysis is used for hemostatic resuscitation of general severe trauma, severe hepatic trauma, severe craniocerebral trauma, and severe trauma with seawater immersion and other types of injuries, has a desirable hemostatic resuscitation effect, does not cause thrombosis in uninjured parts, and can reduce an amplitude of lactate increase after the trauma.

The present disclosure further provides a preparation method of the FDP for well-balanced coagulation and fibrinolysis, including: mixing the components in the parts by mass to obtain the FDP for well-balanced coagulation and fibrinolysis.

As an embodiment, a preparation process of the FDP in the FDP for well-balanced coagulation and fibrinolysis includes the following steps:
mixing a plasma, ascorbic acid, and glycine to obtain a primary plasma;
subjecting the primary plasma to pre-freezing at −40° C. for 2 h, vacuumizing to not greater than 30 Pa, and then freeze-drying at −40° C. for 10 h; and
subjecting a product obtained by the freeze-drying to vacuum-drying at −35° C. for 6 h such that a moisture content of the product is not greater than 3% to obtain the FDP.

As an embodiment, the primary plasma includes the ascorbic acid at a concentration of 2 mmol/L to 4 mmol/L and the glycine at a concentration of 60 mmol/L.

The present disclosure further provides use of the FDP for well-balanced coagulation and fibrinolysis or a FDP for well-balanced coagulation and fibrinolysis prepared by the preparation method in preparation of a hemostatic product.

As an embodiment, the hemostatic product is a hemostatic resuscitation product.

As an embodiment, the hemostatic resuscitation product is used for one or more selected from the group consisting of general severe trauma, severe hepatic trauma, severe craniocerebral trauma, and severe trauma with seawater immersion.

As an embodiment, preparations 1-1, 2, 5-1, and 6 can all be used for general severe trauma (the animal model of Test Example 1). Preparations 3 and 7 can both be used for severe hepatic trauma (the animal model of Test Example 2). Preparations 4 and 8 can both be used for severe craniocerebral trauma (the animal model of Test Example 3). Preparations 1-2 and 5-2 can both be used for severe trauma with seawater immersion (the animal model of Test Example 4).

In the present disclosure, according to different changes in a coagulation-fibrinolysis system after different types of tissue and organ injuries, appropriate concentrations of coagulation factors, anticoagulant factors, fibrinolytic factors, fibrinolysis inhibiting factors, and von Willebrand factor are compounded in FDP. In this way, different preparations of FDP for well-balanced coagulation and fibrinolysis are obtained, which can be used for hemostatic resuscitation of general severe trauma, severe hepatic trauma, severe craniocerebral trauma, and severe trauma with seawater immersion and other types of injuries, with desirable hemostatic resuscitation effects. They do not cause thrombosis in uninjured parts, and may reduce the amplitude of lactate increase after the trauma.

In order to further illustrate the embodiments of the present disclosure, the FDP for well-balanced coagulation and fibrinolysis, the method of preparation and the use thereof provided by the present disclosure are described in detail below with reference to the accompanying drawings and examples, but the accompanying drawings and the examples should not be construed as limiting the protection scope of the present disclosure.

### Example 1

### 1. Basic FDP was Prepared as Follows:

Ascorbic acid with a final concentration of 2 mmol/L to 4 mmol/L (to maintain pH at 7.4 to 7.6) and glycine with a final concentration of 60 mmol/L (to maintain protein stability) were added into fresh plasma, and then freeze-dried as follows. Briefly, the mixture was pre-frozen at −40° C. for 2 h, then pre-vacuumized to not greater than 30 Pa, and freeze-dried at −40° C. for 10 h; after the freeze-drying, the plasma was subjected to vacuum-drying at −35° C. for 6 h to keep the moisture content of the FDP within 3%. The collected and packaged FDP was sterilized by ⁶⁰Co γ ray for later use.

For convenience of preparation, 200 ml of human plasma yields 1 unit of FDP, while for rabbits, based on a relative weight comparison to humans, 20 ml of fresh plasma yields 1 unit of FDP. Both were recorded as the basic FDP.

2. The concentrations of various factors of the coagulation-fibrinolysis system in fresh plasma were determined by ELISA. The results are shown in Table 1.

TABLE 1
Concentrations of factors in normal coagulation-fibrinolysis system

| Factor | Human | Rabbit |
|---|---|---|
| Coagulation factor II | 3 mg/mL | 3.2 mg/mL |
| Fibrinogen | 3.3 g/L | 3.35 g/L |
| Coagulation factor V | 0.08 mg/mL | 0.07 mg/mL |
| Coagulation factor X | 0.08 mg/mL | 0.09 mg/mL |
| Antithrombin III | 0.25 mg/mL | 0.31 mg/mL |
| Plasminogen | 0.12 mg/mL | 0.14 mg/mL |
| Plasminogen activation inhibitor-1 (PAI-1) | 10.0 AU/mL | 10.4 AU/mL |
| Von Willebrand Factor (vWF) | 10.5 μg/mL | 11.2 μg/mL |

3. Changes in the Coagulation-Fibrinolysis System after Different Types of Injury

The following 5 rabbit models were accurately established using the multifunctional trauma-inducing machine developed in the early stage of the laboratory, using built-in infrared aiming and other settings: lower limb fracture+lung injury (injury severity score 18), lower limb fracture+lung injury+intestinal injury+hemorrhagic shock (injury severity score 34), severe hepatic trauma (injury severity score 16), severe penetrating craniocerebral injury (injury severity score 16), and severe soft tissue injury+seawater immersion injury (injury severity score 16). The multifunctional trauma-inducing machine was disclosed in the literature [Ye Z, et al. Characterization of Combined Blast- and Fragment-Induced Pelvic Injuries and Hemostatic Resuscitation in Rabbits. J Surg Res. 2023; 285:158-167.], and the method of use was disclosed in the literature [Du W Q, Zhong X, Jiang R Q, Zong Z W, Jia Y J, Ye Z, Zhou X L. Animal model-based simulation training for three emergent and urgent operations of penetrating thoracic injuries. Chin J Traumatol. 2022 Aug. 2; S1008-1275 (22) 00084-0.]. The method of establishment of different rabbit models was disclosed in the literature [Du W Q, Jiang R Q, Zong Z W, Zhang L, Ye Z, Zhong X, Jia Y J. Establishment of a combat damage control surgery training platform for explosive combined thoraco-abdominal injuries. Chin J Traumatol. 2022 July; 25(4): 193-200.] and [YANG Haoyang, DU Wenqiong, Y E Zhao, et al. Analysis of changes in coagulation-fibrinolysis function in rabbits with soft tissue injury combined with hemorrhagic modification and seawater immersion. Journal of Third Military Medical University 2023; 24 issue]. The injury severity scoring standard could be found in the literature [Du W Q, Jiang R Q, Zong Z W, Zhang L, Ye Z, Zhong X, Jia Y J. Establishment of a combat damage control surgery training platform for explosive combined thoraco-abdominal injuries. Chin J Traumatol. 2022 July; 25(4): 193-200.].

Blood was collected 1 h after injury to measure routine coagulation phase and TEG, and plasma was collected for ELISA to detect the content of coagulation factors, anticoagulant factors, fibrinolytic factors, and antifibrinolytic factors. The results showed that the changes in the coagulation-fibrinolysis system were different in the 5 types of injury.

1 h after injury, the levels of core coagulation factors and anticoagulation factors in patients with lower limb fracture+lung injury were approximately 0.52 to 0.55 times the normal levels, while those in patients with lower limb fracture+lung injury+intestinal injury+hemorrhagic shock decreased further, approximately 0.33 to 0.35 times the normal levels. This result indicated that as the severity of injury increased, the changes in the coagulation-fibrinolysis system became more severe, and a higher multiple of balanced FDP was required for resuscitation.

In the plasma of animals with severe liver injury, the levels of core coagulation factors and anticoagulation factors were similar to those of animals with lower limb fractures+lung injury, but the fibrinogen level dropped to 0.45 g/L, which was about 1/7 of the normal value.

In the plasma of animals with severe craniocerebral penetrating injuries, the levels of core coagulation factors and anticoagulant factors were similar to those of animals with lower limb fractures+lung injuries, but the plasmin level was elevated to about 1.6 times the normal level.

In the plasma of animals with severe soft tissue injury+seawater immersion, the levels of core coagulation factors and anticoagulation factors were similar to those of animals with lower limb fracture+lung injury, but the platelet level decreased more than that of animals with lower limb fracture+lung injury, with a decrease of about 45×10⁹ platelets/L.

### Example 2

A universal FDP for well-balanced coagulation and fibrinolysis included a complementary universal FDP for well-balanced coagulation and fibrinolysis or a 2.0-fold FDP for well-balanced coagulation and fibrinolysis, and a preparation method thereof included: under strict aseptic conditions, the coagulation-fibrinolysis system factors were added to the basic FDP in Example 1 according to the proportions in Table 2, and then mixed and encapsulated.

TABLE 2
Universal FDP for well-balanced coagulation and fibrinolysis

| | Complementary universal | | | | | | | |
|---|---|---|---|---|---|---|---|---|
| | FDP for well-balanced | | | | 2.0-fold FDP for well-balanced | | | |
| / | coagulation and fibrinolysis | | | | coagulation and fibrinolysis | | | |
| Factor | Human | | Rabbit | | Human | | Rabbit | |
| Coagulation factor II | 300 | mg | 32 | mg | 600 | mg | 64 | mg |
| Fibrinogen | 300 | mg | 33.5 | mg | 600 | mg | 67 | mg |
| Coagulation factor V | 8 | mg | 0.7 | mg | 16 | mg | 1.4 | mg |
| Coagulation factor X | 8 | mg | 0.9 | mg | 16 | mg | 1.8 | mg |
| Antithrombin III | 25 | mg | 3.1 | mg | 50 | mg | 6.2 | mg |
| Plasminogen | 12 | mg | 1.4 | mg | 24 | mg | 2.8 | mg |
| PAI-1 | 1000 | AU | 104 | AU | 2000 | AU | 208 | AU |
| VWF | 1.05 | mg | 112 | μg | 2.1 | mg | 224 | μg |

NOTE:
the dosages of various factors in Table 2 were based on 1 unit of basic FDP, and the same applied to Table 3.

### Example 3

As shown in Example 1, when the liver was injured, various coagulation factors synthesized by the liver, especially fibrinogen, were severely reduced; compared with those of conventional injury, the concentrations of plasma plasmin and other substances in severe brain tissue injury were increased, while other changes were the same as those in conventional injury. In view of these changing characteristics, the present disclosure provided a fibrinogen-rich FDP for well-balanced coagulation and fibrinolysis and a fibrinolysis inhibiting factor-rich FDP for well-balanced coagulation and fibrinolysis. The preparation method was similar to that in Example 1, and the amount of coagulation-fibrinolysis system factors added to 1 unit of basic FDP is shown in Table 3.

TABLE 3
Fibrinogen-rich FDP for well-balanced coagulation and fibrinolysis and fibrinolysis
inhibiting factor-rich FDP for well-balanced coagulation and fibrinolysis

| | | | | | Fibrinolysis | | | |
|---|---|---|---|---|---|---|---|---|
| | Fibrinogen-rich | | | | inhibiting factor-rich | | | |
| | coagulation-fibrinolysis | | | | coagulation-fibrinolysis | | | |
| / | high-balance FDP | | | | high-balance FDP | | | |
| Factor | Human | | Rabbit | | Human | | Rabbit | |
| Coagulation factor II | 300 | mg | 32 | mg | 300 | mg | 32 | mg |
| Fibrinogen | 1200 | mg | 134 | mg | 300 | mg | 33.5 | mg |
| Coagulation factor V | 8 | mg | 0.7 | mg | 8 | mg | 0.7 | mg |
| Coagulation factor X | 8 | mg | 0.9 | mg | 8 | mg | 0.9 | mg |
| Antithrombin III | 100 | mg | 12.4 | mg | 25 | mg | 3.1 | mg |
| Plasminogen | 48 | mg | 5.6 | mg | / | | / | |
| PAI-1 | 4000 | AU | 416 | AU | 4000 | AU | 416 | AU |
| VWF | 4.2 | mg | 448 | μg | 4.2 | mg | 448 | μg |

### Example 4

As shown in Example 1, when tissue injury occurred together with seawater immersion, the platelet count decreased more than that without seawater immersion. In view of the observed changes, the present disclosure provided a platelet-rich FDP for well-balanced coagulation and fibrinolysis, and a preparation method thereof was similar to that of the complementary universal FDP for well-balanced coagulation and fibrinolysis in Example 2, except that the basic FDP was replaced with a platelet-rich FDP.

A preparation method of the platelet-rich FDP was similar to that of the basic FDP in Example 1, except that the fresh plasma was replaced with PRP. The platelet-rich FDP obtained from per 200 mL of human PRP was recorded as 1 unit, and the platelet-rich FDP obtained from per 20 mL of rabbit PRP was recorded as 1 unit of FDP.

The preparation method of the PRP included the followings. The whole blood was collected from humans or rabbits, mixed with sodium citrate anticoagulant (4%) at a volume ratio of 1:9, and centrifuged at 450 g for 10 min to obtain a plasma, namely the PRP.

There was no significant difference in appearance between the basic FDP and the 4 different types of FDP prepared in Examples 2 to 4. The moisture content ranged from 3.5% to 4.2%, and there was no significant difference among the 5 FDPs.

### Test Example 1 Experimental Observation on Effect of Universal FDP for Well-Balanced Coagulation and Fibrinolysis

### I. Experimental Method

### 1. Separation of Platelets, Red Blood Cells, and Plasma

After anesthesia, the rabbits were cannulated through their femoral artery and whole blood was collected from the rabbits. The whole blood was mixed with 4% sodium citrate at a volume ratio of 9:1 in a 50 mL centrifuge tube and centrifuged at 3,500 rpm for 20 min and then for another 10 min. The whole blood was separated into three layers, including plasma, platelets (including some white blood cells), and red blood cells from top to bottom, and the plasma and platelets were drawn out for later use.

### 2. In Vitro TEG

Various indicators of in vitro TEG were compared among the normal plasma (Group A), the basic FDP in Example 1 (Group B), the complementary universal FDP for well-balanced coagulation and fibrinolysis in Example 2 (Group C), and the 2.0-fold FDP for well-balanced coagulation and fibrinolysis in Example 2 (Group D).

Briefly, platelets and red blood cells were each added to 5 mL of the 4 types of plasma according to the corresponding proportion of whole blood and mixed. Afterwards, 1 mL of the sample was added into the cup for TEG analysis, which was then added to the coagulation activation reagent tube to allow reaction for 3 min. 20 μL of 0.1 M calcium chloride was added into the cup, and then 340 μL of the sample was taken out from the coagulation activation reagent tube and added into the cup for determination of TEG parameters such as MA. The three indicators of R value, α angle (reflecting the rate of blood clot formation), and MA (reflecting the strength of blood clot) were analyzed in detail referring to a method in the literature [Ye Z, et al. Characterization of Combined Blast- and Fragment-Induced Pelvic Injuries and Hemostatic Resuscitation in Rabbits. J Surg Res. 2023; 285:158-167.].

### 3. Animal Model Preparation and Intervention Measures

2 animal models of lower limb fracture+lung injury (denoted as ISS 18) and lower limb fracture+lung injury+intestinal injury+hemorrhagic shock (denoted as ISS 36) were established using the method in Example 1. At 30 min after injury, the animals were resuscitated by transfusion of FDP-free liquid (Group A), basic FDP (Group B), complementary universal FDP for well-balanced coagulation and fibrinolysis (Group C), and 2.0-fold FDP for well-balanced coagulation and fibrinolysis (Group D). Each group of animals consisted of 8 rabbits. 8 normal rabbits were selected as a normal control group, which was not injured or resuscitated, and only their blood was collected for the following tests, designated as the normal control group (Group NC). The FDP-free liquid was lactated Ringer's solution; the transfusion volume in each group was 10 mL/kg body weight.

### 4. Biochemical Detection Indicators and Detection Methods Thereof

Blood pressure was monitored before injury and 4 h after injury, and the blood was drawn for analysis such as complete blood count, arterial blood gas analysis, and TEG. The hematocrit was recorded using Mindray BC-5180CRP fully automatic blood cell analyzer (Mindray, Shenzhen, China). The i-STAT handheld blood gas analyzer (Flextronics Manufacturing, Singapore) and its supporting CHEM8+ and CG4+ test cards were used to detect the lactate concentration.

TEG detection: 1 mL of whole blood from each group of animals at different time points was collected to allow the same operation and observation as above.

### 5. Thrombosis Detection

The above animals were sacrificed by an overdose of potassium chloride injection 4 h after injury, and then their kidneys, lungs on the uninjured side and other organs were quickly removed. First, gross dissection was conducted to observe the presence of thrombosis with the naked eye. Then, the organs were fixated with conventional 4% paraformaldehyde. The tissues were embedded, and sections were sliced, followed by HE staining to observe the presence of ectopic thrombosis.

### II. Experimental Results

1. The in vitro TEG results are shown in FIG. 1 to FIG. 3.

Compared with normal plasma, the R value of the basic FDP group was significantly increased, the α angle was smaller than that of normal plasma, and the MA value was significantly lower than that of normal plasma. In the complementary universal FDP for well-balanced coagulation and fibrinolysis group and the 2.0-fold FDP for well-balanced coagulation and fibrinolysis group, the R value was significantly shorter than that of normal plasma and basic FDP, the α angle was significantly larger than that of the other two groups, the MA value was significantly higher than that of the other two groups, and a change trend of the 2.0-fold FDP for well-balanced coagulation and fibrinolysis was more obvious than that of the complementary universal FDP for well-balanced coagulation and fibrinolysis.

2. The coagulation indicator detection results of the in vivo experiment are shown in FIG. 4 to FIG. 6.

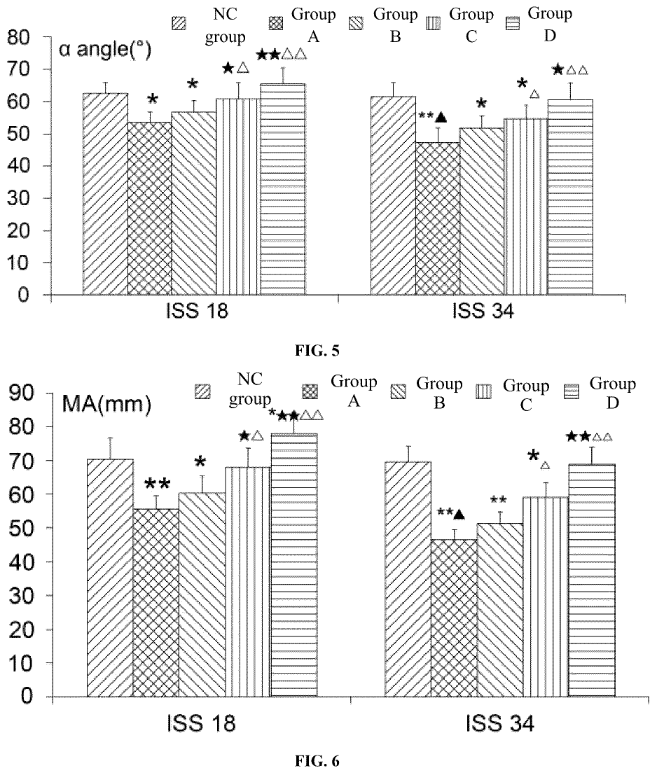

After injury, the R values of the animals in the ISS 18 group and the ISS 34 group were significantly higher than those of the normal animals, and the R value of the animals in the ISS 34 group was significantly higher than that of the animals in the ISS 18 group (FIG. 4). The α angle and MA value also showed the same trend (FIG. 5 and FIG. 6). This suggested that as the severity of the injury increased, the coagulation disorder became more severe, manifested by a more obvious prolongation of the R value and a more obvious decrease in the α angle and MA value.

In the ISS 18 group, the complementary universal FDP for well-balanced coagulation and fibrinolysis could restore to normal values better than the basic FDP, and the R value of the 2.0-fold FDP for well-balanced coagulation and fibrinolysis was significantly lower than the normal value (FIG. 4). The results suggested that for animals with injury severity of around 18, the complementary universal FDP for well-balanced coagulation and fibrinolysis was sufficient to correct the R value to normal, while the 2.0-fold FDP for well-balanced coagulation and fibrinolysis resulted in an excessively significant shortening of the R value, which was an overcorrection. This phenomenon also showed a similar trend in the changes of MA value and a angle (FIG. 5 and FIG. 6).

In the ISS 34 group, although the complementary universal FDP for well-balanced coagulation and fibrinolysis restored the R value better than the basic FDP, it was still significantly different from the normal value. The 2.0-fold FDP for well-balanced coagulation and fibrinolysis restored the R value to a level close to the normal level, which was not significantly different from the normal value (FIG. 4). The results suggested that for animals with injury severity of about 34, the 2.0-fold FDP for well-balanced coagulation and fibrinolysis could correct the R value to a close normal level. Similar trend also appeared in the changes of MA value and α angle (FIG. 5 and FIG. 6).

3. The results of blood loss are shown in FIG. 7.

The results of blood loss calculated by hematocrit showed that in animals with lower limb fractures+lung injury, the complementary universal FDP for well-balanced coagulation and fibrinolysis could significantly reduce blood loss compared with the basic FDP and the 2.0-fold FDP for well-balanced coagulation and fibrinolysis, and there was no significant difference between the two (FIG. 7). The results suggested that it was not necessary to use a 2.0-fold FDP for well-balanced coagulation and fibrinolysis in animals with moderate injuries, such as lower limb fractures and lung injuries.

In animals with lower limb fracture+lung injury+intestinal injury+hemorrhagic shock, although the complementary universal FDP for well-balanced coagulation and fibrinolysis was better at reducing blood loss than the basic FDP, its blood loss was significantly higher than that of the 2.0-fold FDP for well-balanced coagulation and fibrinolysis (FIG. 7). The results suggested that high-balance FDP was required in animals with severe injuries such as lower limb fractures+lung injury+intestinal injury+hemorrhagic shock.

4. The results of lactate changes are shown in FIG. 8.

The lactate concentration in each injury group was significantly increased. The conventional FDP resulted in a certain decrease in lactate concentration, but there was no significant difference compared with the control group without plasma transfusion. However, the balance FDP resulted in a further decrease in lactate concentration, which was significantly different from the group without plasma transfusion.

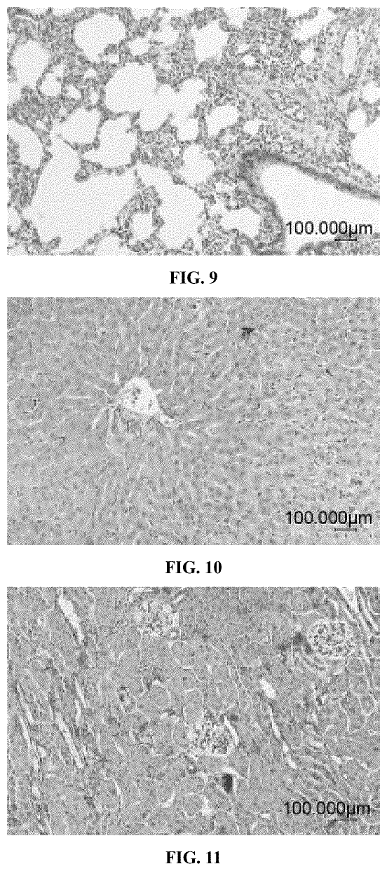

5. The results of thrombus detection are shown in FIG. 9 to FIG. 11.

H&E staining showed that no thrombosis was present in the kidneys, lungs, livers and other organs of the animals in each group.

### Test Example 2 Experimental Observation on Effects of Fibrinogen-Rich FDP for Well-Balanced Coagulation and Fibrinolysis

### I. Experimental Method

1. Separation of platelets, red blood cells, and plasma was conducted according to the method in Test Example 1.

2. The in vitro TEG was similar to that in Test Example 1, except that the 2.0-fold FDP for well-balanced coagulation and fibrinolysis in Group D was replaced with the fibrinogen-rich FDP for well-balanced coagulation and fibrinolysis in Example 3.

3. Animal model preparation and intervention measures

The animal model of severe hepatic trauma was established by the method in Example 1. At 30 min after injury, the animals were resuscitated by transfusion of FDP-free liquid, basic FDP, complementary universal FDP for well-balanced coagulation and fibrinolysis, and fibrinogen-rich FDP for well-balanced coagulation and fibrinolysis, denoted as Groups A to D. Each group of animals consisted of 8 rabbits. 8 normal rabbits were selected as a normal control group, which was not injured or resuscitated, and only their blood was collected for the following tests, designated as the normal control group (Group NC). The FDP-fee liquid was lactated Ringer's solution; the transfusion volume in each group was 10 mL/kg body weight.

4. Biochemical detection indicators and detection methods thereof were the same as those in Test Example 1.

5. Thrombosis detection was the same as that in Test Example 1.

### II. Experimental Results

1. The in vitro TEG results are shown in FIG. 12 to FIG. 14.

Compared with that of normal plasma, the R value of the basic FDP group was significantly increased, the α angle was smaller than that of normal plasma, and the MA value was significantly lower than that of normal plasma. The R value of the complementary universal FDP for well-balanced coagulation and fibrinolysis group and the fibrinogen-rich FDP for well-balanced coagulation and fibrinolysis group was significantly shorter than that of normal plasma and basic FDP, the α angle was significantly larger than that of the other two groups, and the MA value was significantly higher than that of the other two groups. Moreover, the MA value of the fibrinogen-rich FDP for well-balanced coagulation and fibrinolysis group was significantly higher than that of the complementary universal FDP for well-balanced coagulation and fibrinolysis group, while there was no significant difference in the R value and α angle between the two groups.

2. The coagulation indicator detection results of the in vivo experiment are shown in FIG. 15 to FIG. 17.

The results of in vivo experiment showed that in an animal model of severe liver injury, the basic FDP could better restore various coagulation and fibrinolysis indicators, but there was a large gap with normal values. However, except for the MA value, which did not restore to the normal level, the coagulation indicators of the complementary universal FDP for well-balanced coagulation and fibrinolysis were basically close to the normal values with no significant difference. The fibrinogen-rich FDP for well-balanced coagulation and fibrinolysis made various indicators including MA basically return to normal levels, showing no significant differences when compared to normal values.

3. The results of blood loss are shown in FIG. 18.

The results of blood loss calculated by hematocrit showed that the fibrinogen-rich FDP for well-balanced coagulation and fibrinolysis had the least blood loss, which was less than the complementary universal FDP for well-balanced coagulation and fibrinolysis and the basic FDP, and this was related to the effective improvement of MA value. The blood loss of the complementary universal FDP for well-balanced coagulation and fibrinolysis was less than that of the basic FDP.

4. The results of lactate concentration are shown in FIG. 19.

In this model, lactate rose substantially after injury and was significantly higher than normal lactate concentrations. The complementary universal FDP for well-balanced coagulation and fibrinolysis could partially reduce the lactate concentration, which was significantly lower than that of the basic FDP and the group not using FDP, while the fibrinogen-rich FDP for well-balanced coagulation and fibrinolysis could further reduce the lactate concentration, which was lower than that of the basic FDP and the complementary universal FDP for well-balanced coagulation and fibrinolysis.

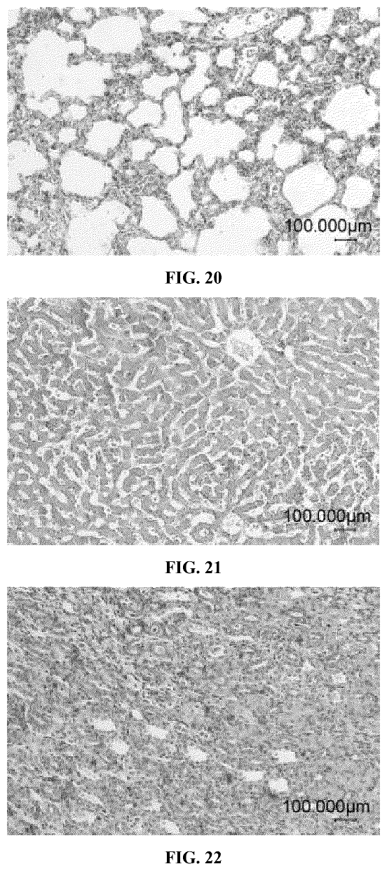

5. The results of thrombus detection are shown in FIG. 20 to FIG. 22.

H&E staining suggested that no thrombosis was found in the kidneys, lungs, livers and other organs of the animals in each group.

### Test Example 3 Experimental Observation on Effects of Fibrinolysis Inhibiting Factor-Rich FDP for Well-Balanced Coagulation and Fibrinolysis

### I. Experimental Method

1. Separation of platelets, red blood cells, and plasma was conducted according to the method in Test Example 1.

2. The in vitro TEG was similar to that in Test Example 1, except that the 2.0-fold FDP for well-balanced coagulation and fibrinolysis in Group D was replaced with the fibrinolysis inhibiting factor-rich FDP for well-balanced coagulation and fibrinolysis in Example 3.

3. Animal model preparation and intervention measures

The animal model of severe craniocerebral trauma was established by the method in Example 1. At 30 min after injury, the animals were resuscitated by transfusion of FDP-free liquid, basic FDP, complementary universal FDP for well-balanced coagulation and fibrinolysis, and fibrinolysis inhibiting factor-rich FDP for well-balanced coagulation and fibrinolysis, denoted as Groups A to D. Each group of animals consisted of 8 rabbits. 8 normal rabbits were selected as a normal control group, which was not injured or resuscitated, and only their blood was collected for the following tests, recorded as the normal control group (Group NC). The group without FDP transfusion was transfused with lactated Ringer's solution; a transfusion volume in each group was 10 mL/kg body weight.

4. Biochemical detection indicators and detection methods thereof were the same as those in Test Example 1.

5. Thrombosis detection was the same as that in Test Example 1.

### II. Experimental Results

1. The in vitro TEG results are shown in FIG. 23 to FIG. 25.

Compared with normal plasma, the R value of the basic FDP group was significantly increased, the α angle was smaller than that of normal plasma, and the MA value was significantly lower than that of normal plasma. The R value of the complementary universal FDP for well-balanced coagulation and fibrinolysis group and the fibrinolysis inhibiting factor-rich FDP for well-balanced coagulation and fibrinolysis group was significantly shorter than that of the basic FDP group, the α angle was significantly larger than that of the basic FDP group, and the MA value was significantly higher than that of the basic FDP group. There were no significant differences in R value, α angle, and MA value between the complementary universal FDP for well-balanced coagulation and fibrinolysis group and the fibrinolysis inhibiting factor-rich FDP for well-balanced coagulation and fibrinolysis group.

2. The coagulation indicator detection results of the in vivo experiment are shown in FIG. 26 to FIG. 28.

In Group A where FDP was not used for resuscitation, the R value, α angle, and MA after injury were significantly different from normal values. Basic FDP could restore various coagulation and fibrinolysis indicating factors to a certain extent, but there was a large gap with normal values. The R value, α angle, and MA values were significantly different from normal values. The R value, α angle, and MA value of Group B animals resuscitated with basic FDP were better than those of Group A animals resuscitated with FDP, but there was no statistical difference.

The indicators of the complementary universal FDP for well-balanced coagulation and fibrinolysis group and the fibrinolysis inhibiting factor-rich FDP for well-balanced coagulation and fibrinolysis group were close to normal values. The R value, α angle, and MA value were not significantly different from the normal values, and were significantly better than those of Group A without FDP resuscitation. Compared with the animals in Group B resuscitated with basic FDP, the R value, α angle, and MA value of the complementary universal FDP for well-balanced coagulation and fibrinolysis group and the fibrinolysis inhibiting factor-rich FDP for well-balanced coagulation and fibrinolysis group were significantly better than those of Group B.

The indicators of the complementary universal FDP for well-balanced coagulation and fibrinolysis group and the fibrinolysis inhibiting factor-rich FDP for well-balanced coagulation and fibrinolysis group were similar, with no significant differences.

3. The results of blood loss are shown in FIG. 29.

The results of blood loss calculated by hematocrit showed that there was the least blood loss of the fibrinolysis inhibiting factor-rich FDP for well-balanced coagulation and fibrinolysis, which was significantly less than that of the non-FDP resuscitation group. This might be because the rich content of fibrinolysis inhibiting factor inhibited thrombolysis, thereby reducing bleeding volume.

The blood loss in the complementary universal FDP for well-balanced coagulation and fibrinolysis group was significantly lower than that in the non-FDP resuscitation group. The blood loss of the complementary universal FDP for well-balanced coagulation and fibrinolysis was less than that of the basic FDP, but showed no significant difference.

4. The results of lactate concentration are shown in FIG. 30.

In this model, lactate rose substantially after injury and was significantly higher than normal lactate concentrations. The basic FDP could slightly reduce the lactate concentration, which had no significant difference from the non-FDP resuscitation group and was still significantly higher than the normal value.

After complementary FDP use, lactate concentration was significantly lower than that in the no-FDP resuscitation group. The soft tissue concentration of the fibrinolysis inhibiting factor-rich FDP for well-balanced coagulation and fibrinolysis group was further reduced and significantly lower than that of Groups A, B and C, which was related to the fact that the bleeding volume was the least among all groups.

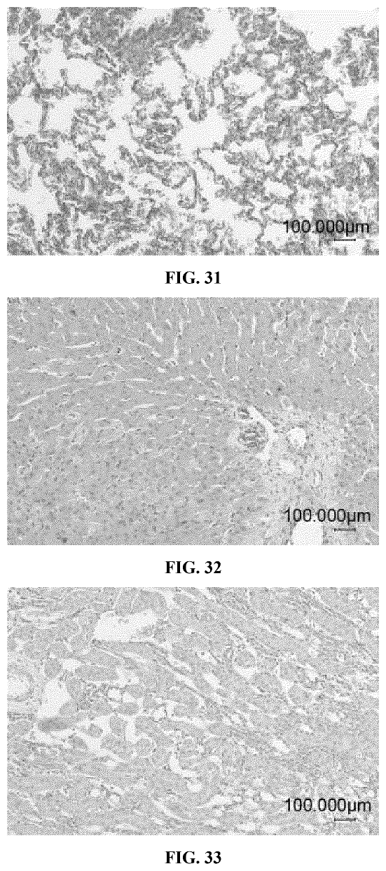

5. The results of thrombus detection are shown in FIG. 31 to FIG. 33.

H&E staining suggested that no thrombosis was found in the kidneys, lungs, livers and other organs of the animals in each group.

### Test Example 4 Observation Experiment on the Effect of Platelet-Rich FDP for Well-Balanced Coagulation and Fibrinolysis

### I. Experimental Method

1. Separation of platelets, red blood cells, and plasma was conducted according to the method in Test Example 1.

2. The in vitro TEG was similar to that in Test Example 1, except that the 2.0-fold FDP for well-balanced coagulation and fibrinolysis in Group D was replaced with the platelet-rich FDP for well-balanced coagulation and fibrinolysis in Example 4.

3. Animal model preparation and intervention measures

An animal model of severe soft tissue injury was established by the method of Example 1, and the animals were immediately immersed in simulated seawater for 10 min after injury. 30 min after injury induction, the animals were resuscitated by transfusion of FDP-free liquid, basic FDP, complementary universal FDP for well-balanced coagulation and fibrinolysis, and platelet-rich FDP for well-balanced coagulation and fibrinolysis, denoted as Groups A to D. Each group of animals consisted of 8 rabbits. 8 normal rabbits were selected as a normal control group, which was not injured or resuscitated, and only their blood was collected for the following tests, recorded as the normal control group (Group NC). The FDP-free liquid was lactated Ringer's solution; a transfusion volume in each group was 10 mL/kg body weight.

4. Biochemical detection indicators and detection methods thereof were the same as those in Test Example 1.

5. Thrombosis detection was the same as that in Test Example 1.

### II. Experimental Results

1. The in vitro TEG results are shown in FIG. 34 to FIG. 36.

Compared with normal plasma, the R value of the basic FDP group was significantly increased, the α angle was smaller than that of normal plasma, and the MA value was significantly lower than that of normal plasma. In the complementary universal FDP for well-balanced coagulation and fibrinolysis group and the platelet-rich FDP for well-balanced coagulation and fibrinolysis group, the R value was significantly shorter than that of the basic FDP group, and the α angle was significantly larger than that of the basic FDP group.

Compared with the platelet-rich FDP for well-balanced coagulation and fibrinolysis group, there were no significant differences in R value and α angle between the two groups, but the MA value of the platelet-rich FDP for well-balanced coagulation and fibrinolysis group was significantly higher than that of the complementary universal FDP for well-balanced coagulation and fibrinolysis group, which was related to the high platelet content in the platelet-rich FDP for well-balanced coagulation and fibrinolysis.

2. The coagulation indicator detection results of the in vivo experiment are shown in FIG. 37 to FIG. 39.

In Group A where FDP was not used for resuscitation, the R value, α angle, and MA after injury were significantly different from normal values. Basic FDP could restore various coagulation and fibrinolysis indicators to a certain extent, but there was a large gap with normal values. The R value, α angle, and MA values were significantly different from normal values. The R value, α angle, and MA value of Group B animals resuscitated with basic FDP were better than those of Group A animals resuscitated without FDP.

There were no significant differences in R value and α angle between the platelet-rich FDP for well-balanced coagulation and fibrinolysis group (Group D) and the complementary universal FDP for well-balanced coagulation and fibrinolysis group (Group C), but the MA value of the former was significantly higher than that of the latter.

3. The results of blood loss are shown in FIG. 40.

The results of blood loss calculated by hematocrit showed that the platelet-rich FDP for well-balanced coagulation and fibrinolysis had the least blood loss, which was less than the complementary universal FDP for well-balanced coagulation and fibrinolysis and the basic FDP, and this was related to its effective improvement of MA value and thus the quality of thrombosis. The blood loss of the complementary universal FDP for well-balanced coagulation and fibrinolysis was less than that of the basic FDP.

4. The results of lactate concentration are shown in FIG. 41.

In this model, lactate rose substantially after injury and was significantly higher than normal lactate concentrations. The basic FDP could slightly reduce the lactate concentration, which had no significant difference from the non-FDP resuscitation group and was still significantly higher than the normal value.

After the use of complementary universal FDP for well-balanced coagulation and fibrinolysis, lactate concentration was significantly lower than that in the no-FDP resuscitation group. The soft tissue concentration of the platelet-rich FDP for well-balanced coagulation and fibrinolysis group was further reduced and significantly lower than that of Groups A, B and C, which was related to the fact that the bleeding volume was the least among all groups.

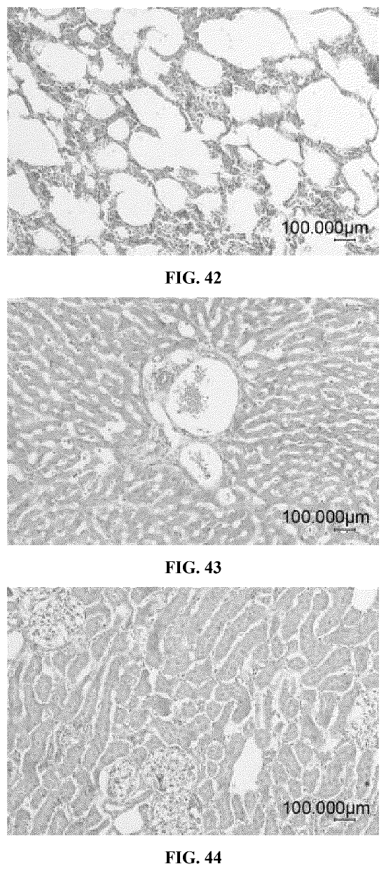

5. The results of thrombus detection are shown in FIG. 42 to FIG. 44.

H&E staining suggested that no thrombosis was found in the kidneys, lungs, livers and other organs of the animals in each group.

In conclusion, the FDP for well-balanced coagulation and fibrinolysis is used for hemostatic resuscitation of general severe trauma, severe hepatic trauma, severe craniocerebral trauma, and severe trauma with seawater immersion and other types of injuries, and demonstrates a desirable hemostatic resuscitation effect.

Although the above examples have illustrated the present disclosure in detail, it is only a part of, not all of, the embodiments of the present disclosure. Other embodiments may also be obtained by persons based on the example without creative efforts, and all of these examples shall fall within the protection scope of the present disclosure.

What is claimed is:

1. A coagulation-fibrinolysis high-balance freeze-dried plasma (FDP), comprising the following components in parts by mass: an FDP, 32 parts to 600 parts of coagulation factor II, 33.5 parts to 1,200 parts of fibrinogen, 0.7 parts to 16 parts of coagulation factor V, 0.9 parts to 16 parts of coagulation factor X, 3.1 parts to 50 parts of antithrombin III, 0 parts to 24 parts of plasminogen, plasminogen activation inhibitor-1, and 0.112 parts to 2.1 parts of von Willebrand factor; wherein the ratio of a mass of coagulation factor II to an enzyme activity of the plasminogen activation inhibitor-1 is (32-600) mg: (104-4000) AU; and assuming the FDP prepared from 200 mL of a human plasma or 20 mL of a rabbit plasma is recorded as 1 unit, the FDP and the coagulation factor II are at a ratio of 1 unit: (32-600) mg.

2. The FDP for well-balanced coagulation and fibrinolysis according to claim 1, wherein the FDP for well-balanced coagulation and fibrinolysis is one or more selected from the group consisting of independently packaged
preparation 1, comprising the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; wherein the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2, comprising the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; wherein the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3, comprising the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4, comprising the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
wherein the FDP in the preparation 1 to the preparation 4 is human FDP;
preparation 5, comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; wherein the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6, comprising the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; wherein the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7, comprising the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg; and
preparation 8, comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg;
wherein the FDP in the preparation 5 to the preparation 8 is rabbit FDP.

3. The FDP for well-balanced coagulation and fibrinolysis according to claim 2, wherein a raw material of the FDP in the preparation 1 is selected from the group consisting of a fresh human plasma and a human platelet-rich plasma (PRP); and a raw material of the FDP in the preparation 5 is selected from the group consisting of fresh rabbit plasma and rabbit PRP.

4. The FDP for well-balanced coagulation and fibrinolysis according to claim 2, wherein a raw material of the FDP in the preparation 2 to the preparation 4 is fresh human plasma; and a raw material of the FDP in the preparation 6 to the preparation 8 is fresh rabbit plasma.

5. A method for preparing the FDP for well-balanced coagulation and fibrinolysis according to claim 1, comprising: mixing the components in the parts by mass to obtain the FDP for well-balanced coagulation and fibrinolysis.

6. The method according to claim 5, wherein the FDP for well-balanced coagulation and fibrinolysis is one or more selected from the group consisting of independently packaged
preparation 1 comprising the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; wherein the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2 comprising the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; wherein the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3 comprising the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4 comprising the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
wherein the FDP in the preparation 1 to the preparation 4 is human FDP;
preparation 5 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; wherein the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6 comprising the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; wherein the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7 comprising the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg; and
preparation 8 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg;
wherein the FDP in the preparation 5 to the preparation 8 is rabbit FDP.

7. The method according to claim 6, wherein a raw material of the FDP in the preparation 1 is selected from the group consisting of a fresh human plasma and a human platelet-rich plasma (PRP); and a raw material of the FDP in the preparation 5 is selected from the group consisting of a fresh rabbit plasma and a rabbit PRP.

8. The method according to claim 6, wherein a raw material of the FDP in the preparation 2 to the preparation 4 is fresh human plasma; and a raw material of the FDP in the preparation 6 to the preparation 8 is fresh rabbit plasma.

9. The method according to claim 5, wherein a preparation process of the FDP in the FDP for well-balanced coagulation and fibrinolysis comprises the following steps:
mixing a plasma, ascorbic acid, and glycine to obtain a primary plasma;
subjecting the primary plasma to pre-freezing at −40° C. for 2 h, vacuumizing to not greater than 30 Pa, and then freeze-drying at −40° C. for 10 h; and
subjecting a product obtained by the freeze-drying to vacuum-drying at −35° C. for 6 h such that a moisture content of the product is not greater than 3% to obtain the FDP.

10. The method according to claim 9, wherein the primary plasma comprises the ascorbic acid at a concentration of 2 mmol/L to 4 mmol/L and the glycine at a concentration of 60 mmol/L.

11. A method for treating trauma, comprising administering to a subject in need thereof a hemostatic product comprising the FDP for well-balanced coagulation and fibrinolysis according to claim 1.

12. The method according to claim 11, wherein the hemostatic product is a hemostatic resuscitation product.

13. The method according to claim 12, wherein the trauma is selected from the group consisting of general severe trauma, severe hepatic trauma, severe craniocerebral trauma, and severe trauma with seawater immersion.

14. The method according to claim 11, wherein the FDP for well-balanced coagulation and fibrinolysis is one or more selected from the group consisting of independently packaged
preparation 1 comprising the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; wherein the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2 comprising the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; wherein the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3 comprising the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4 comprising the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
wherein the FDP in the preparation 1 to the preparation 4 is human FDP;
preparation 5 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; wherein the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6 comprising the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; wherein the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7 comprising the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg; and
preparation 8 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg;
wherein the FDP in the preparation 5 to the preparation 8 is rabbit FDPpreparation 1 comprising the following components in parts by mass: an FDP, 300 parts of coagulation factor II, 300 parts of fibrinogen, 8 parts of coagulation factor V, 8 parts of coagulation factor X, 25 parts of antithrombin III, 12 parts of plasminogen, plasminogen activation inhibitor-1, and 1.05 parts of von Willebrand factor; wherein the ratio of a mass of coagulation factor II to an enzyme activity of the plasminogen activation inhibitor-1 is 300 mg: 1000 AU; and the FDP and the coagulation factor II are at a ratio of 1 unit: 300 mg;
preparation 2 comprising the following components in parts by mass: an FDP, 600 parts of coagulation factor II, 600 parts of fibrinogen, 16 parts of coagulation factor V, 16 parts of coagulation factor X, 50 parts of antithrombin III, 24 parts of plasminogen, plasminogen activation inhibitor-1, and 2.1 parts of von Willebrand factor; wherein the ratio of a mass of coagulation factor II to an enzyme activity of the plasminogen activation inhibitor-1 is 600 mg: 2000 AU; and the FDP and the coagulation factor II are at a ratio of 1 unit: 600 mg;
preparation 3 comprising the following components in parts by mass: an FDP, 300 parts of coagulation factor II, 1,200 parts of fibrinogen, 8 parts of coagulation factor V, 8 parts of coagulation factor X, 100 parts of antithrombin III, 48 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
preparation 4 comprising the following components in parts by mass: an FDP, 300 parts of coagulation factor II, 300 parts of fibrinogen, 8 parts of coagulation factor V, 8 parts of coagulation factor X, 25 parts of antithrombin III, 0 parts of plasminogen, plasminogen activation inhibitor-1, and 4.2 parts of von Willebrand factor; wherein the ratio of a mass of coagulation factor II to an enzyme activity of the plasminogen activation inhibitor-1 is 300 mg: 4000 AU; and the FDP and the coagulation factor II are at a ratio of 1 unit: 300 mg;
wherein the FDP in the preparation 1 to the preparation 4 is human FDP;
preparation 5 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 1.4 parts of plasminogen, plasminogen activation inhibitor-1, and 0.112 parts of von Willebrand factor; wherein the ratio of a mass of coagulation factor II to an enzyme activity of the plasminogen activation inhibitor-1 is 32 mg: 104 AU; and the FDP and the coagulation factor II are at a ratio of 1 unit: 32 mg;
preparation 6 comprising the following components in parts by mass: an FDP, 64 parts of coagulation factor II, 67 parts of fibrinogen, 1.4 parts of coagulation factor V, 1.8 parts of coagulation factor X, 6.2 parts of antithrombin III, 2.8 parts of plasminogen, plasminogen activation inhibitor-1, and 0.208 parts of von Willebrand factor; wherein the ratio of a mass of coagulation factor II to an enzyme activity of the plasminogen activation inhibitor-1 is 64 mg: 2084 AU; and the FDP and the coagulation factor II are at a ratio of 1 unit: 64 mg;
preparation 7 comprising the following components in parts by mass: an FDP, 32 parts of coagulation factor II, 134 parts of fibrinogen, 0.7 parts of coagulation factor V, 0.9 parts of coagulation factor X, 12.4 parts of antithrombin III, 5.6 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg; and
preparation 8 comprising the following components in parts by mass: an FDP, 32 parts of coagulation factor II, 33.5 parts of fibrinogen, 0.7 parts of coagulation factor V, 0.9 parts of coagulation factor X, 3.1 parts of antithrombin III, 0 parts of plasminogen, plasminogen activation inhibitor-1, and 0.448 parts of von Willebrand factor; wherein the ratio of a mass of coagulation factor II to an enzyme activity of the plasminogen activation inhibitor-1 is 32 mg: 416 AU; and the FDP and the coagulation factor II are at a ratio of 1 unit: 32 mg;
wherein the FDP in the preparation 5 to the preparation 8 is rabbit FDP.

15. The method according to claim 14, wherein a raw material of the FDP in the preparation 1 is selected from the group consisting of a fresh human plasma and a human platelet-rich plasma (PRP); and a raw material of the FDP in the preparation 5 is selected from the group consisting of a fresh rabbit plasma and a rabbit PRP.

16. The method according to claim 14, wherein a raw material of the FDP in the preparation 2 to the preparation 4 is fresh human plasma; and a raw material of the FDP in the preparation 6 to the preparation 8 is fresh rabbit plasma.
